(19) United States
(12) Patent Application Publication
(10) Pub. No.: US 2006/0137105 A1
(43) Pub. Date: Jun. 29, 2006

(54) Drying control apparatus and method of washing and drying machine

(75) Inventors: Sang Wook Hong, Seoul (KR); Jong Min Lee, Kyungki-do (KR)

Correspondence Address: BIRCH STEWART KOLASCH & BIRCH, PO BOX 747, FALLS CHURCH, VA, 22040-0747, US

(73) Assignee: LG Electronics Inc., Seoul (KR)

(21) Appl. No.: 11/269,807

(22) Filed: Nov. 9, 2005

(30) Foreign Application Priority Data
Nov. 12, 2004 (KR) 2004-92560
Nov. 25, 2004 (KR) 2004-97561
Nov. 25, 2004 (KR) 2004-97562

(51) Int. Cl. D06F 29/00; A01H 5/02

(52) U.S. Cl. 8/147; 68/19.2; 68/20

(57) ABSTRACT

A drying control method and apparatus of a washing and drying machine, in which cooling water dropped from a condensation duct to a tub accelerates the condensation of moisture contained in circulated air so as to improve condensing capability, a cold air drying time is differently set according to load so that when the load is small, a drying time is shortened, and the drying of laundry is rapidly performed, a power consumption rate is minimized, or a cooling water consumption rate is minimized according to user's desire, and a drying operation is controlled using a difference between a temperature of air and a temperature of cooling water so that the drying operation is efficiently performed according to load conditions so as to improve the drying capability.

### BACKGROUND OF THE INVENTION

1. Field of the Invention

The present invention relates to a drying machine or a washing and drying machine, and more particularly to a drying control apparatus and method of a washing and drying machine which enable a drying operation to be appropriately performed according to operating conditions.

2. Description of the Related Art

General washing machines are divided into washing machines only having washing function, drying machines only having drying function, and washing and drying machines having washing and drying functions.

FIG. 1 is a longitudinal sectional view illustrating the internal structure of a conventional washing and drying machine.

The conventional washing and drying machine, as shown in FIG. 1, comprises a tub 10 installed in a cabinet 2 and supported by springs 4 and dampers 6, a drum 20 installed in the tub 10 for containing laundry, a door 22 connected to the cabinet 2 for opening and closing the front surface of the drum 20, a heater duct 30 installed on the tub 10 for discharging hot air to the tub 10 and having a heater 26 and an air blower 27 installed therein, and a condensation duct 40, one end of which is connected to the lower part of a side surface of the tub 10, and, the other end of, which is connected to the heater duct 30, for condensing moisture in circulated air.

A gasket 12, which contacts the door 22 when the door 22 is closed, is installed on the tub 10. The heater duct 30 is connected to the gasket 12.

A motor 14 for rotating the drum 20 is installed on the tub 10.

A water supply device 15 for supplying washing water or rinsing water to the inside of the tub 10 in a washing operation or a rinsing operation is connected to the tub 10. A drainage device 16 is connected to the lower part of the tub 10.

The drainage device 16 comprises a drainage bellows 17 connected to the lower part of the tub 10, a drainage pump 18 connected to the drainage bellows 17, and a drainage hose 19 connected to the drainage pump 18.

Through holes 22 for passing washing water or air are formed through the circumferential surface or the rear surface of the drum 20.

Lifters 24 for moving laundry are installed on the inner circumferential surface of the drum 20.

The air blower 27 comprises a circulation fan 28 rotatably disposed in the heater duct 30, and a fan motor 29 installed in the heater duct 30 for rotating the circulation fan 28.

A cooling water supply unit 42 for supplying cooling water to the inside of the condensation duct 40 so that moisture in the circulated air obtained by drying the laundry is condensed is connected to the condensation duct 40.

The cooling water supply unit 42 comprises a cooling water valve 44 connected to an external hose 43 for intermitting the cooling water supplied from the external hose 43, and a cooling water hose 45 for guiding the cooling water having passed through the cooling water valve 44 to the inside of the condensation duct 40.

Hereinafter, the operation of the above conventional washing and drying machine will be described.

First, when the washing and drying machine is operated under the condition that laundry is placed in the drum 20 and the door 22 is closed, washing water is supplied to the washing and drying machine through the water supply device 15.

The washing water is supplied to the inside of the tub 10 and is contained in the tub 10. Then, the washing water flows to the inside of the drum 20 through the through holes 22 of the drum 20 so that the laundry in the drum 20 is submerged in the washing water.

When the motor 14 is operated after the supply of the washing water is completed, the drum 20 is rotated, and the laundry in the drum 20 moves, thereby being washed by means of the function of the washing water.

After the above washing operation is completed, the contaminated washing water in the tub 10 is discharged to the outside of the washing and drying machine through the drainage device 16.

Thereafter, a rinsing operation for eliminating foam remaining in the laundry is repeated several times. In the same manner as the washing operation, in the rinsing operation, the water supply device 15 and the motor 14 are controlled so that foam remaining in the laundry is eliminated, and the contaminated water containing the foam is discharged to the outside of the washing and drying machine through the drainage device 16.

After the repetition of the rinsing operation, a dehydrating operation for centrifugally dehydrating the laundry is performed.

That is, the motor 14 is driven at a high speed so that the laundry is centrifugally dehydrated, and the water obtained by dehydrating the laundry is discharged to the outside of the washing and drying machine through the drainage device 16.

Thereafter, a drying operation for drying the laundry is performed.

First, the motor 14 is driven to rotate the drum 20, and the laundry in the drum 20 is agitated.

The heater 26 is switched on to increase the temperature of peripheral air, the fan motor 29 is driven to rotate the circulation fan 28, and the cooling water valve 44 and the drainage pump 18 are repeatedly switched on and off at a designated time interval.

The air in the drum 20 contacts the laundry by the rotation of the circulation fan 18, and is converted into a low-temperature and high-humidity state by receiving heat and moisture of the laundry. Then, the air in the low-temperature and high-humidity state flows to a space between the drum 20 and the tub 10 through the through holes 22, and is introduced into the condensation duct 40. When the air passes through the condensation duct 40, moisture contained in the air is condensed by cooling water in the condensation duct 40.

When the air, having passed through the condensation duct 40, passes through the heater duct 30, the air is heated by the heater 26 and is converted into hot air. The hot air passes through the gasket 12, is discharged to the inside of the gasket 12, and is circulated into the drum 20. Thereafter, the above circulation is repeated, thereby drying the laundry.

The cooling water, which is supplied to the condensation duct 40, passes through the condensation duct 40, is collected in the tub 10, and is periodically pumped by the drainage pump 18, thereby being discharged to the outside.

After the above drying of the laundry using hot air is performed for a designated time, the heater 26 is switched off, and the drum 20 and the circulation fan 28 are circulated so that drying of the laundry using cold air is performed.

After the above drying of the laundry using cold air is performed for a designated time, the circulation fan 28 and the motor 14 are switched off. Thereby, the drying operation is completed.

Since the cooling water is periodically supplied to the conventional washing and drying machine, and discharged to the outside regardless of the load and the temperature of the cooling water, the cooling water consumption rate and the electric power consumption rate are increased. Further, the drying of the laundry using cold air is performed at a designated time, when the load of the laundry is small, the overall drying time is lengthened, and when the load of the laundry is large, the laundry is not sufficiently cooled, thereby causing users to be burned.

Moreover, since the cooling water is supplied to the conventional washing and drying machine, and discharged to the outside at a designated time interval in the drying operation, the temperature of peripheral air and the temperature of the cooling water are not compensated for. Thus, the conventional washing and drying machine cannot cope with various load conditions, and has a limit in efficiently performing the drying operation.

### SUMMARY OF THE INVENTION

Therefore, the present invention has been made in view of the above problems, and it is an object of the present invention to provide a drying control method of a washing and drying machine, in which cooling water dropped from a condensation duct to a tub accelerates the condensation of moisture contained in circulated air, thereby improving condensing capability and minimizing a cooling water consumption rate and a power consumption rate.

It is a further object of the present invention to provide a drying control method of a washing and drying machine, in which a drying time using cold air is differently set according to load, and, when the load is small, a drying time is shortened, and when the load is large, a drying efficiency is increased and burns of users are prevented.

It is another object of the present invention to provide a drying control method of a washing and drying machine, in which the drying of laundry is rapidly performed, a power consumption rate is minimized, or a cooling water consumption rate is minimized according to user's desire.

It is yet another object of the present invention to provide a drying control apparatus and method of a washing and drying machine, in which a drying operation is controlled by determining the dryness of laundry using a difference between a temperature of air and a temperature of cooling water so that the drying operation is efficiently performed according to load conditions, such as the temperature of the air or the temperature of the cooling water, thereby improving the drying capability.

In accordance with a first aspect of the present invention, the above and other objects can be accomplished by the provision of a drying control method of a washing and drying machine, in which air in a drum is circulated so that the air sequentially passes through a tub, a condensation duct, and a heater duct, and is introduced again to the inside of the drum, and dries laundry in the drum, comprising: supplying cooling water for condensing moisture contained in the circulated air to the condensation duct; measuring a temperature of the cooling water dropped from the condensation duct to the tub, or a temperature of the circulated air; and discharging the cooling water from the tub to the outside of the tub, when the measured temperature is more than a designated temperature.

In accordance with a second aspect of the present invention, there is provided a drying control method of a washing and drying machine comprising: drying laundry in a drum using hot air; and drying the laundry in the drum using cold air after the drying of the laundry using the hot air, wherein, in the drying of the laundry using the cold air, the circulation of air is terminated when a temperature of the circulation air is less than a designated cold air drying termination temperature.

In accordance with a third aspect of the present invention, there is provided a drying control method of a washing and drying machine, which controls a circulation fan for causing air in a drum to sequentially pass through a tub, a condensation duct, and a heater duct and to be introduced again into the drum, a cooling water valve for supplying cooling water to the condensation duct so that moisture contained in the air passing through the condensation duct is condensed by the cooling water, and a heater for heating the air passing through the heater duct, wherein: ON/OFF conditions of the circulation fan, the cooling water valve, and the heater are determined according to the input of kind, drying time, and dryness of laundry to be dried, and when one mode out of a rapid drying mode, a power saving mode, and a water saving mode is inputted to the washing and drying machine, the ON/OFF condition of at least one of the circulation fan, the cooling water valve, and the heater is changed according to the inputted mode.

In accordance with a fourth aspect of the present invention, there is provided a drying control apparatus of a washing and drying machine comprising: a tub having a drum installed therein for containing laundry so that the laundry is washed or dried; a condensation duct connected to one side of the tub for eliminating moisture contained in air drying the laundry by condensation; a heater duct connected to the upper end of the condensation duct and the other side of the tub for heating the air having passed through the condensation duct and circulating the air into the tub; an air temperature sensor for measuring a temperature of the air dehumidified by passing through the condensation duct; a cooling water temperature sensor installed at the lower end of the tub or at an inlet of the condensation duct for measuring the temperature of cooling water; and control means for receiving results sensed by the air temperature sensor and the cooling water temperature sensor and controlling the drying of the laundry using a difference between the temperature of the air and the temperature of the cooling water.

In accordance with a fourth aspect of the present invention, there is provided a drying control apparatus of a washing and drying machine, in which: when air drying laundry passes through a condensation duct after a drying operation for drying the laundry is started, a temperature of cooling -water condensing moisture contained the air and a temperature of the air dehumidified by passing through the condensation duct are measured, and when the drying operation is continued, the dried state of the laundry is determined by a difference temperature between a signal representing characteristics of the temperature of the cooling water and a signal representing characteristics of the temperature of the air, so as to control the drying operation.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a longitudinal sectional view illustrating the internal structure of a conventional washing and drying machine;

FIG. 2 is a longitudinal sectional view illustrating the internal structure of a washing and drying machine in accordance with the present invention;

FIG. 3 is another longitudinal sectional view illustrating the internal structure of the washing and drying machine in accordance with the present invention;

FIG. 4 is a sectional view of another installation state of a cooling water temperature sensor of FIG. 3;

FIG. 5 is a control block diagram of a washing and drying machine, to which a drying control method in accordance with a first embodiment of the present invention is applied;

FIG. 6 is a flow chart illustrating the drying control method in accordance with the first embodiment of the present invention;

FIG. 7 is a graph illustrating water supply/drainage times of the drying control method in accordance with the first embodiment of the present invention;

FIG. 8 is a control block diagram of a washing and drying machine, to which a drying control method in accordance with a second embodiment of the present invention is applied;

FIG. 9 is a flow chart illustrating the drying control method in accordance with the second embodiment of the present invention;

FIG. 10 is a flow chart illustrating the drying control method in accordance with the second embodiment of the present invention in one mode out of a rapid drying mode, a power saving mode, and a water saving mode.

FIG. 11 is a graph illustrating cooling water supply/discharge times and the temperature of the cooling water or circulated air according to a cold air drying operation when the drying control method in accordance with the second embodiment of the present invention is in one mode out of the rapid drying mode, the power saving mode, and the water saving mode;

FIG. 12 is a control block diagram of a washing and drying machine, to which a drying control method in accordance with a third embodiment of the present invention is applied;

FIG. 13 is a graph illustrating temperature characteristics of air and cooling water in the drying control method in accordance with the third embodiment of the present invention;

FIG. 14 is a graph illustrating temperatures sensed by sensors and differences among the sensed temperatures in an initial stage of a drying operation of the drying control method in accordance with the third embodiment of the present invention;

FIG. 15 is a graph illustrating temperatures sensed by sensors and differences among the sensed temperatures in a continuous drying operation in the drying control method in accordance with the third embodiment of the present invention; and

FIG. 16 is a graph illustrating the functional relation among amount of laundry, dryness of the laundry according to drying intensity, and time in the washing and drying machine in accordance with the third embodiment of the present invention.

### DESCRIPTION OF THE PREFERRED EMBODIMENTS

Now, preferred embodiments of the present invention will be described in detail with reference to the annexed drawings.

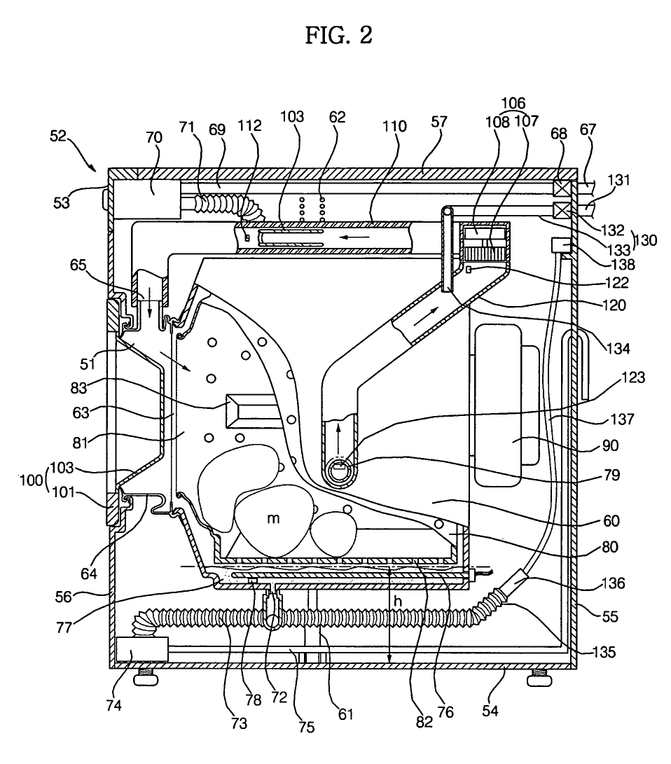

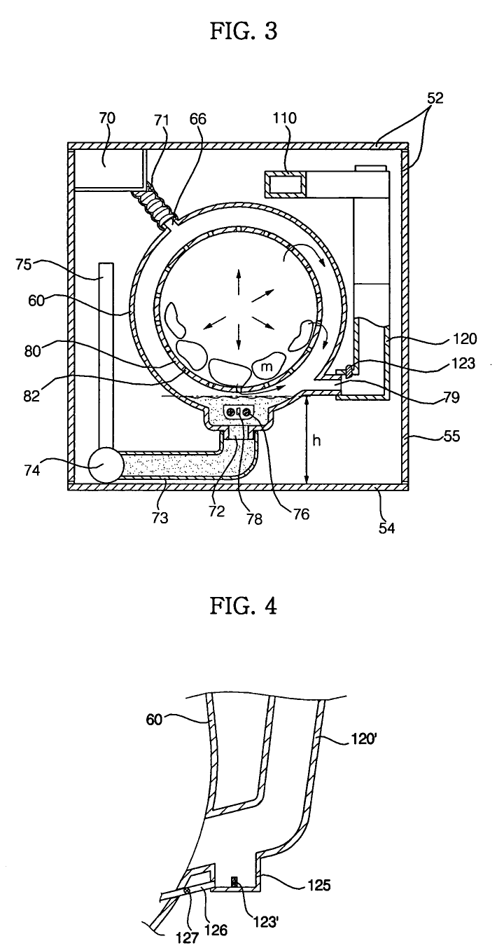

FIG. 2 is a longitudinal sectional view illustrating the internal structure of a washing and drying machine in accordance with the present invention. FIG. 3 is another longitudinal sectional view illustrating the internal structure of the washing and drying machine in accordance with the present invention. FIG. 4 is a sectional view of another installation state of a cooling water temperature sensor of FIG. 3.

As shown in FIGS. 2 and 3, the washing and drying machine of the present invention comprises a cabinet 52 provided with a laundry entrance hole for placing or taking laundry (m) into and out of the machine, a tub 60 installed in the cabinet 52, a drum 80 installed in the tub 60 for containing the laundry, a motor 90 installed on the tub 60 for supporting/rotating the drum 80, a door 100 connected to the cabinet 52 for opening and closing the front surface of the drum 80, a heater duct 110 installed above the tub 60 for discharging hot air to the tub 60 and having a heater 103 and an air blower 106 provided therein, a condensation duct 120 connected to the tub 60 and the heater duct 110 for condensing moisture of air, and a cooling water supplier 130 for supplying cooling water for condensing the moisture of the air to the condensation duct 120.

The cabinet 52 forms the external appearance of the washing and drying machine, and a control panel 53 for controlling various operations of the washing and drying machine and time is installed on the upper portion of the front surface or the upper surface of the cabinet 52.

The cabinet 52 comprises a base pan 54, a cabinet main body 55 installed on the base pan 54, a cabinet cover 56 installed in front of the cabinet main body 55, and a top plate 57 installed on the cabinet main body 55.

The tub 60 is installed in the cabinet 52 using dampers 61 and springs 62, which are connected to the cabinet 52, so that impact applied to the tub 60 can be absorbed.

An opening 63 is formed through the tub 60 in the rear of the laundry entrance hole 51, and the tub 60 is horizontally installed in the cabinet 52.

A gasket 64, which is installed on the opening 63 and the laundry entrance hole 51 and contacts the door 10 when the door 100 is closed, is provided on the tub 60.

A duct communication hole 65, to which the heater duct 100 is connected, is protruded from the gasket 64.

A water supply hole 66 is formed through one side of the upper portion of the tub 60, and a water supply device is connected to the water supply hole 66.

The water supply device comprises a water supply valve 68 installed in the cabinet 52 and connected to an external hose 67 for intermitting water supplied through the external hose 67, a water supply hose 69 connected to the water supply valve 68 for guiding the water having passed through the water supply valve 68, a detergent storage 70 connected to the water supply hose 69 so that the water guided by the water supply hose 69 is mixed with a detergent and having a water supply channel, a detergent receiving portion, and a discharge hole, and a water supply bellows 71 connected to the detergent storage 70 and the water supply hole 66 for guiding washing water, which is mixed with the detergent, having passed through the detergent storage 70 or rinsing water, which is not mixed with the detergent, to the water supply hole 66 of the tub 60.

A drainage hole 72 is formed through one side of the lower portion of the tub 60, and a drainage device is connected to the drainage hole 72.

The drainage device comprises a drainage bellows 73 connected to the drainage hole 72 of the tub 60, a drainage pump 74 connected to the drainage bellows 73, and a drainage hose 75 connected to the drainage pump 74 and extended to the outside of the washing and drying machine.

A washing heater 76 for heating the washing water in the tub 60 in a washing operation is installed in the tub 60. A heater-receiving groove 77 for receiving the washing heater 76 is formed in the central area of the bottom of the tub 60.

A tub temperature sensor 78 for measuring the temperature of the washing heater 76 so as to interrupt current applied to the washing heater 76 when the washing heater 76 is superheated is installed in the washing heater 76.

A duct communication hole 79, to which the condensation duct 120 of the tub 60 is connected, is formed through the side of the tub 60.

A through hole, through which a drive shaft of the motor 90 passes, is formed through the rear surface of the tub 60.

An opening 81 is formed through the drum 80 in the rear of the laundry entrance hole 51, and the drum 80 is horizontally installed in the tub 60.

Through holes 82 are formed through the circumferential surface or the rear surface of the drum 80.

Lifters 83 for moving the laundry are installed on the inner circumferential surface of the drum 80.

The motor 90 comprises a stator installed on the rear surface of the tub 60, a rotor rotated by magnetic force formed between the stator and the rotor, and the drive shaft axially installed on the rotor, passing through the through hole formed through the rear surface of the tub 60, and connected to the rear surface of the drum 80.

The door 100 comprises a door frame 101 rotatably connected to the cabinet 52 and provided with an opening formed through the central area thereof, and a door glass 103 installed on the door frame 101 and protruded from the door frame 101 towards the drum 80.

The air blower 106 comprises a circulation fan 107 rotatably disposed in the heater duct 110, and a fan motor 108 installed on the heater duct 110 for rotating the circulation fan 107.

A heater air temperature sensor 112 for measuring the temperature the air heated by the heater 103 to prevent the laundry (m) from being damaged due to the overheating of the air circulating to the drum 80 is installed in the heater duct 110.

A condensed air temperature sensor 122 for measuring the temperature of the air condensed by the cooling water through the condensation duct 120 to control a drying operation is installed in the upper portion of the condensation duct 120. Preferably, the condensed air temperature sensor 122 is installed between an inlet of the circulation fan 107 and a cooling water inlet 134 so as to appropriately measure the temperature of the air without contacting drops of the cooling water, thereby measuring the temperature of the air sufficiently exchanging heat with the cooling water.

A cooling water temperature sensor 123 or 123′ is installed in the lower part of the condensation duct 120 at a position just after the duct communication hole 79. As shown in FIGS. 2 and 3, the cooling water temperature sensor 123 may be installed at the upper portion of the inside of the lower part of the condensation duct 120, thereby not contacting cooling water so that the cooling water temperature sensor 123 is not influenced by water drops generated by the condensation of moisture contained in air. Further, as shown in FIG. 4, the cooling water temperature sensor 123′ may be installed in a cooling water chamber 125 installed under the condensation duct 120′ so that the cooling water temperature sensor 123′ is submerged in the cooling water.

Here, the cooling water chamber 125 comprises a hose 126 and a valve 127 for discharging the cooling water so as to prevent the cooling water from being collected in the cooling water chamber 125.

The cooling water supplier 130 comprises a cooling water valve 132 connected to an external hose 131 for intermitting the cooling water supplied through the external hose 131, and a cooling water hose 133 for guiding the cooling water having passed through the cooling water valve 132 to the inside of the condensation duct 120.

A water level sensing unit for sensing the water level in the tub 60 is installed in the washing and drying machine.

The water level sensing unit comprises a water level sensing bellows 135 connected to one end of the drainage bellows 73, an air chamber 136 having a lower end connected to the water level sensing bellows 135 and filled with air compressed according to the level of water filling the water level sensing bellows 135, a water level sensing tube 137 having a lower end connected to one end of the air chamber 136, and a water level sensor 138, to which the upper end of the water level sensing tube 137 is connected, for sensing the water level by sensing the pressure of the air in the water level sensing tube 137.

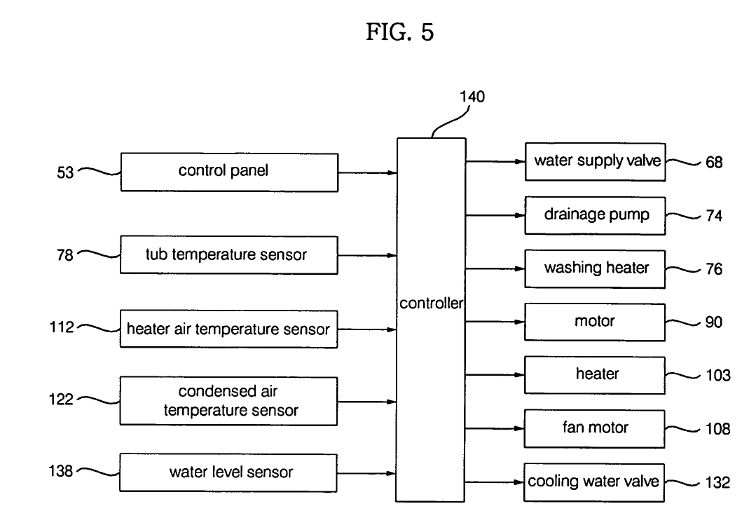

FIG. 5 is a control block diagram of a washing and drying machine, to which a drying control method in accordance with a first embodiment of the present invention is applied.

The washing and drying machine further comprises a controller 140 for controlling the water supply valve 68, the drainage pump 74, the washing heater 76, the motor 90, the heater 103, the fan motor 108, and the cooling water valve 132 according to instructions inputted via the control panel 53.

That is, when washing and rinsing instructions are inputted via the control panel 53, the controller 140 controls the water supply valve 68, the motor 90, the drainage pump 74, and the washing heater 76 so that washing and rinsing operations are performed.

When dehydrating instructions are inputted via the control panel 53, the controller 140 controls the motor 90 and the drainage pump 74 so that a dehydrating operation is performed.

When drying instructions are inputted via the control panel 53, the controller 140 controls the motor 90 and the heater 103, and controls the drainage pump 74, the fan motor 108, and the cooling water valve 132 according to a temperature (T) sensed by at least one of the tub temperature sensor 78, the heater air temperature sensor 112, and the condensed air temperature sensor 122 for forming automatic drying algorithm. Thereby, a drying operation is performed.

The controller 140 may use the temperature (T) sensed by any one of the tub temperature sensor 78, the heater air temperature sensor 112, and the condensed air temperature sensor 122, and may use the temperatures (T) sensed by all of the tub temperature sensor 78, the heater air temperature sensor 112, and the condensed air temperature sensor 122. Hereinafter, the tub temperature sensor 78 will be used in an initial or middle stage of the drying operation (i.e. in a hot air drying mode), and one of the heater air temperature sensor 112 and the condensed air temperature sensor 122, particularly, the heater air temperature sensor 112, will be used in a last stage of the drying operation (i.e. in a cold air drying mode).

The drying control method of the washing and drying machine in accordance with the first embodiment of the present invention will be described, as below.

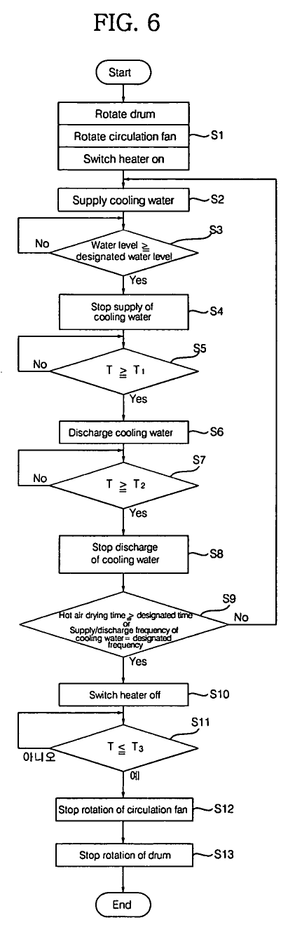

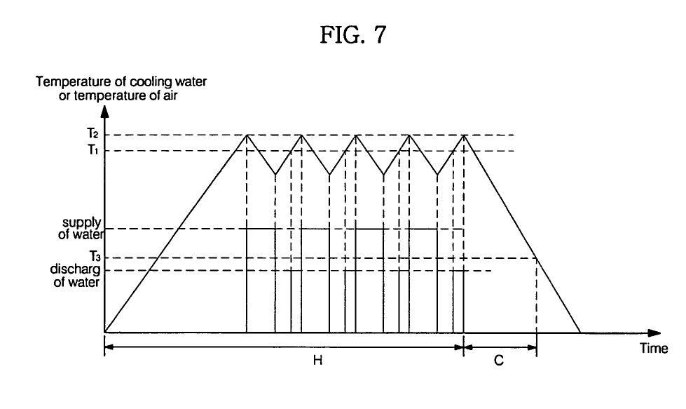

FIG. 6 is a flow chart illustrating the drying control method of the washing and drying machine in accordance with the first embodiment of the present invention, and FIG. 7 is a graph illustrating water supply/drainage times of the drying control method of the washing and drying machine in accordance with the first embodiment of the present invention.

In the drying operation of the washing and drying machine, as shown in FIG. 6, the hot air drying mode (H) is first performed, and the cold air drying mode (C) is then performed. When the cold air drying mode (C) is completed, the overall drying operation is terminated.

In the hot air drying mode (H), the controller 140, as shown in FIGS. 2 to 5, drives the motor 90 so that the drum 80 is rotated to agitate the laundry (m) in the drum 80, drives the fan motor 108 so that the circulation fan 107 is rotated to cause the air in the drum 80 to sequentially pass through the through holes 82 of the drum 80, the tub 60, the condensation duct 120, and the heater duct 110 and to be circulated into the drum 80, and switches the heater 103 on so that the air passing through the heater duct 110 is heated by the heater 103 (S1).

Thereafter, the controller 140 switches the cooling water valve 132 on so that the cooling water is supplied to the condensation duct 120 and moisture contained in the air passing through the condensation duct 120 is condensed by the cooling water (S2).

As shown in FIG. 2, the air in the drum 80 (denoted by the dotted line) moves towards the through holes 82 by the rotation of the circulation fan 107, contacts the laundry (m) agitated in the drum 80, and robs the laundry (m) of moisture, thereby being converted into a low-temperature and high-humidity state. After the air passes through the through holes 82, the air moves to a space between the outer cylindrical surface of the drum 80 and the inner cylindrical surface of the tub 60, and then moves to the condensation duct 120.

The air in the low-temperature and high-humidity state moved to the condensation duct 120 is robbed of heat by the cooling water dropped from the condensation duct 120 so that moisture contained in the air is condensed into water, moves to the condensation duct 110, and is heated by the heater 103 in the condensation duct 110, thereby being converted into a high-temperature and low-humidity state.

The air in the high-temperature and low-humidity state passes through the gasket 64, and is circulated into the drum 80.

When the cooling water supplied by the cooling water valve 132 after the cooling water valve 132 is switched on flows to the tub 60 and reaches a designated water level (h) in the tub 60, the controller 140 switches the cooling water valve 132 off so that the supply of the cooling water is stopped (S3 and S4).

Here, the designated water level (h), as shown in FIG. 3, is a water level limit of the cooling water dropping from the condensation duct 120, which is contained in the tub 60. The designated water level (h) is set to be higher than the inner lower surface of the tub 60, i.e., the upper end of the drainage hole 72 so that moisture contained in the air moving to the lower portion of the inside of the tub 60 is condensed by the cooling water contained in the lower portion of the inside of the tub 60, and to be lower than the outer lower surface of the drum 80 so that the cooling water contained in the lower portion of the inside of the tub 60 is not introduced into the drum 80 through the through holes 82 of the drum 80.

After the supply of the cooling water is stopped, as shown in FIG. 3, among the air in the high-humidity state (denoted in a solid line) having passed through the through holes 82 of the drum 80, a part of the air, which passes between the outer lower surface of the drum 80 and the inner lower surface of the tub 60 is robbed of heat by the cooling water contained in the lower portion of the inside of the tub 60, and moisture contained in the part of the air is condensed into water. Then, the part of the air is joined with the other part of the air, which does not pass between the outer lower surface of the drum 80 and the inner lower surface of the tub 60, and the joined air is introduced into the condensation duct 120.

The air, which is introduced into the condensation duct 120, passes through the condensation duct 120, and is introduced into the heater duct 110. When the air passes through the heater duct 110, the air is heated by the heater 103, and is converted into a high-temperature and low-humidity state. Then, the air in the high-temperature and low-humidity state passes through the gasket 64, and is circulated into the drum 80.

As time passes, the temperature of the circulated air (denoted in the solid line) or the cooling water contained in the lower portion of the inside of the tub 60 is increased according to the decrease in heat absorption rate/ moisture condensation rate of the cooling water, as shown in FIG. 7.

The controller 140 compares the temperature (T) measured by the tub temperature sensor 78 to a first designated temperature (T₁), and, when the temperature (T) is more than the first designated temperature (T₁), switches the drainage pump 74 on (S5 and S6).

When the drainage pump 74 is switched on, the cooling water contained in the lower portion of the inside of the tub 60 is discharged to the outside of the washing and drying machine through the drainage hose 75.

After the drainage pump 74 is switched on, the controller 140 compares the temperature (T) measured by the tub temperature sensor 78 to a second designated temperature (T₂), and, when the temperature (T) is more than the second designated temperature (T₂), switches the drainage pump 74 off (S7 and S8).

When the drainage pump 74 is switched off, the discharge of the cooling water is stopped.

Thereafter, when it is determined that the time of the hot air drying mode (H) does not elapse a designated hot air drying time or the supply/discharge frequency of the cooling water does not reach a designated frequency, the controller 140 switches the cooling water valve 132 on for re-supplying the cooling water, thereby re-supplying the cooling water to the washing and drying machine (S9 and S2).

When the cooling water is re-supplied to the washing and drying machine, in heat absorption rate/moisture condensation rate of the re-supplied cooling water is increased, and the temperature (T) measured by the tub temperature sensor 78 is decreased, as shown in FIG. 6.

When the level of the re-supplied cooling water reaches a designated water level, the controller 140 switches the cooling water valve 132 off again, thereby stopping the re-supply of the cooling water (S3 and S4).

Thereafter, the controller 140 compares the temperature (T) measured by the tub temperature sensor 78 to the first designated temperature (T₁), and switches the drainage pump 74 on according to the compared results so that the cooling water is re-discharged to the outside of the washing and drying machine. Then, the controller 140 compares the temperature (T) measured by the tub temperature sensor 78 to the second designated temperature (T₂), and switches the drainage pump 74 off according to the compared results so that re-discharge of the cooling water is stopped. Further, the controller 140 compares the time of the hot air drying mode to the designated hot air drying time or the supply/discharge frequency of the cooling water to the designated frequency, and repeats the supply/discharge of the cooling water according to the compared results (S5, S6, S7, S8, and S9).

When it is determined that the time of the hot air drying mode (H) elapses the designated hot air drying time or the supply/discharge frequency of the cooling water reaches the designated frequency, the controller 140 switches the heater 103 off, thereby performing the cold air drying mode (C) (S9 and S10).

In the cold air drying mode (C), the heater 103 is switched off, and only the circulation fan 107 is continuously rotated. Then, the air in the drum 80 is circulated into the tub 60, the condensation duct 120, the heater duct 110, and the drum 80, thus gradually lowering the temperature of the laundry (m).

The controller 140 compares the temperature (T) measured by the heater air temperature sensor 112 to a designated cold air drying termination temperature (T₃) so that the cold air drying time is adjusted according to load, and, when the temperature (T) is less than the designated cold air drying termination temperature (T₃), switches the fan motor 108 off, thereby terminating the circulation of the air in the drum 80 (S11 and S12).

Here, the designated cold air drying termination temperature (T₃) is a temperature for determining whether or not the cold air drying mode (C) is terminated.

That is, when the load is small, it takes a short time to reach the designated cold air drying termination temperature (T₃), and when the load is large, it takes a long time to reach the designated cold air drying termination temperature (T₃). Accordingly, whether or not the cold air drying mode (C) is terminated is determined by the designated cold air drying termination temperature (T₃). When the load is small, the drying time is shortened, and when the load is large, the drying efficiency is increased and burns of consumers are prevented.

Further, the controller 140 switches the motor 90 off, thereby terminating the overall drying operation including the cold air drying mode.

Since the cooling water is not discharged just after the supply but is contained in the tub, so that moisture contained in the circulated air is condensed by the cooling water in the tub, the drying control method of the washing and drying machine in accordance with the first embodiment of the present invention improves condensing efficiency and minimizes an overall drying time and a power consumption rate.

Further, since the cooling water is discharged when the temperature of the cooling water contained in the tub or the temperature of the circulated air is increased more than a designated temperature, the drying control method of the washing and drying machine in accordance with the first embodiment of the present invention minimizes a cooling water consumption rate.

Moreover, since the cold air drying mode in the last stage of the drying operation is terminated according to the load, the drying control method of the washing and drying machine in accordance with the first embodiment of the present invention shortens the drying time when the load is small, and increases the drying efficiency and prevents burns of consumers when the load is large.

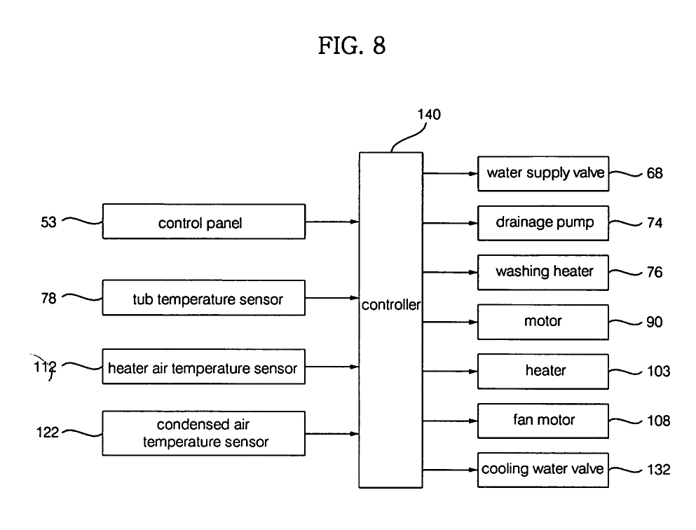

FIG. 8 is a control block diagram of a washing and drying machine, to which a drying control method in accordance with a second embodiment of the present invention is applied.

The washing and drying machine further comprises the controller 140 for controlling the water supply valve 68, the drainage pump 74, the washing heater 76, the motor 90, the heater 103, the fan motor 108, and the cooling water valve 132 according to washing, rinsing, dehydrating and drying instructions inputted via the control panel 53.

When washing and rinsing instructions are inputted via the control panel 53, the controller 140 controls the water supply valve 68, the motor 90, the drainage pump 74, and the washing heater 76 so that washing and rinsing operations are performed.

When dehydrating instructions are inputted via the control panel 53, the controller 140 controls the motor 90 and the drainage pump 74 so that a dehydrating operation is performed.

When kind (for controlling the drying temperature), drying time, and dryness of laundry to be dried are inputted via the control panel 53, the controller 140 performs a drying operation according to drying conditions suitable for the inputted data. Then, when one mode out of a rapid drying mode, a power saving mode, and a water saving mode is additionally inputted via the control panel 53, the controller 140 can perform the drying operation by changing the drying conditions according to the inputted mode. Further, when one mode out of the rapid drying mode, the power saving mode, and the water saving mode is additionally inputted via the control panel 53 regardless of the input of the kind, the drying time, and the dryness of laundry to be dried, the controller 140 can perform the drying operation according to the inputted mode.

Hereinafter, the drying operation is performed according to the input of one mode out of the rapid drying mode, the power saving mode, and the water saving mode as well as the input of the kind, the drying time, and the dryness of laundry to be dried.

That is, the controller 140 controls the switching on/off of the motor 90, the drainage pump 74, the heater 103, the fan motor 108, and the cooling water valve 132 so that the overall drying time is maximally shortened in the rapid drying mode, controls the switching on/off of the motor 90, the drainage pump 74, the heater 103, the fan motor 108, and the cooling water valve 132 so that the power consumption rate is minimized in the power saving mode, and controls the switching on/off of the motor 90, the drainage pump 74, the heater 103, the fan motor 108, and the cooling water valve 132 so that the water consumption rate is minimized in the water saving mode.

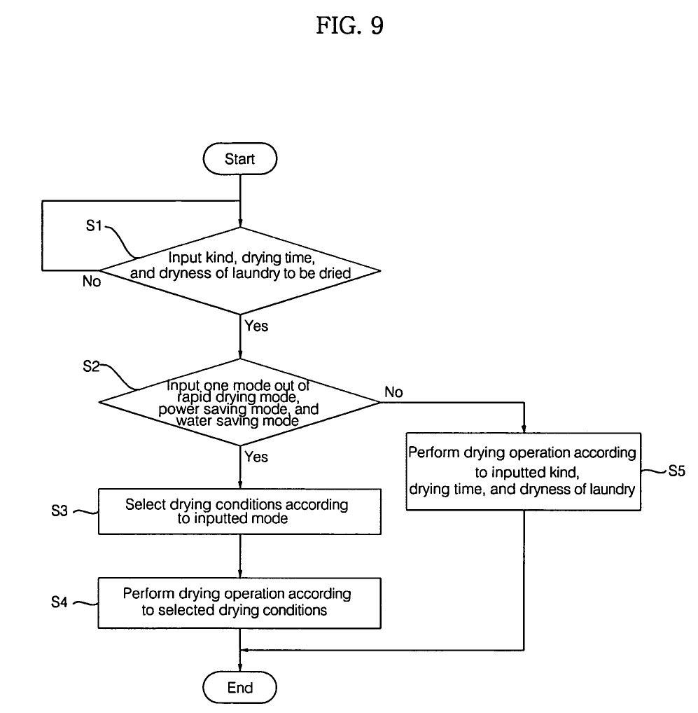

FIG. 9 is a flow chart illustrating the drying control method of the washing and drying machine in accordance with the second embodiment of the present invention.

When a user inputs the kind, the drying time, and the dryness of laundry to be dried via the control panel 53, the controller 140 sets drying conditions according to the inputted kind, drying time, and dryness of the laundry (Si).

When the user inputs one mode out of the rapid drying mode, the power saving mode, and the water saving mode after the input of the kind, the drying time, and the dryness of the laundry, as described above, the controller 140 changes the drying conditions according to the inputted mode and sets the changed drying conditions as new drying conditions (S2 and S3).

The controller 140 switches the motor 90, the drainage pump 74, the heater 103, the fan motor 108, and the cooling water valve 132 on and/or off according to the set new drying conditions, thereby performing the drying operation (S4).

On the other hand, when the user does not input any mode out of the rapid drying mode, the power saving mode, and the water saving mode after the input of the kind, the drying time, and the dryness of laundry to be dried, as described above, the controller 140 switches the motor 90, the drainage pump 74, the heater 103, the fan motor 108, and the cooling water valve 132 on and/or off according to general drying conditions based on the kind, the drying time, and the dryness of laundry to be dried (S2 and S5).

Hereinafter, drying conditions of the rapid drying mode, the power saving mode, and the water saving mode will be described in more detail.

As described in the below Table 1, in the rapid drying mode, the power saving mode, and water saving mode, at least one of an initial cooling water supply time, a cooling water supply cycle, a cooling water discharge cycle, and a cold air drying time is set to a different value.

TABLE 1

| | Initial | Cooling | Cooling | Cold air | | Power | Water |
|---|---|---|---|---|---|---|---|
| | cooling water | water supply | water discharge | drying | Drying | consumption | consumption |
| Mode | supply time | cycle | cycle | time | time | rate | rate |
| Rapid drying mode | 1 | 1 | 1 | 1 | 100 | 106 | 310 |
| Power saving mode | 1 | 2 | 3 | 2 | 104 | 100 | 166 |
| Water saving mode | 2 | 3 | 3 | 1 | 108 | 106 | 100 |

The cooling water serves to dehumidify the circulated air. However, since the cooling water decreases the temperatures of the drum, the tub, and the heater duct (hereinafter, collectively referred to a “system”), the cooling water is not necessary until the inside of the drum is fully dried due to the increase in the temperature of the system more than a designated temperature, and causes heat loss.

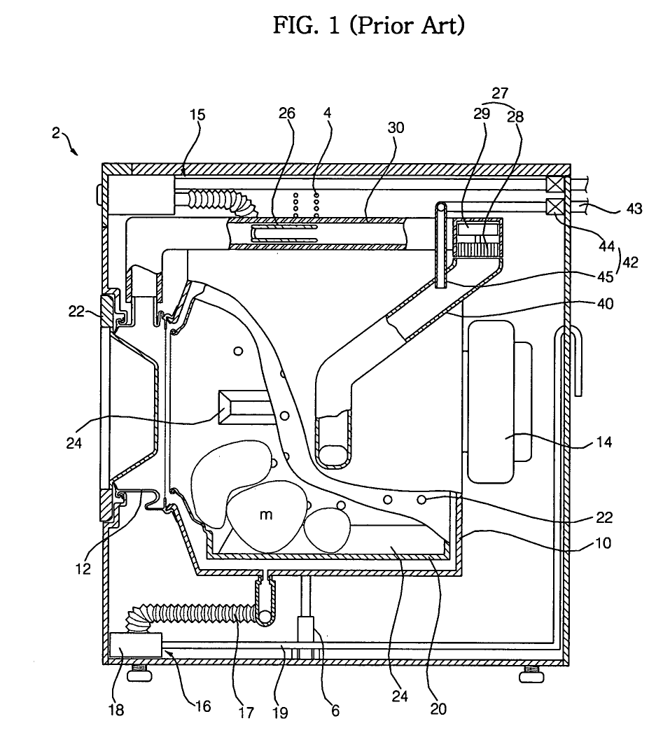

Preferably, the initial supply of the cooling water is performed when the temperature of the system is more than a designated temperature. As shown in FIG. 1, in the rapid drying mode and the power saving mode, the initial supply of the cooling water is performed relatively early so that the drying time is minimized or the power consumption rate is minimized, and in the water saving mode, the initial supply of the cooling water is performed relatively late so that the water consumption rate is minimized.

The supply cycle (ON/OFF) of the cooling water relates to the drying time, the power consumption rate and the water consumption rate. Accordingly, as the supply amount of the cooling water is increased, the drying time is shortened and the power consumption rate and the water consumption rate are increased.

That is, in the rapid drying mode, the cooling water supply cycle is the most shortened to shorten the drying time so that the power consumption rate and the water consumption rate are increased. In the power saving mode, the cooling water supply cycle is longer than that in the rapid drying mode so that the drying time is relatively increased and the power consumption rate and the water consumption rate are decreased. In the water saving mode, the cooling water supply cycle is the longest so that the drying time is the most increased and the power consumption rate and the water consumption rate are decreased.

The discharge cycle (ON/OFF) of the cooling water relates to the time when the cooling water is collected in the tub. Since the circulated air is dehumidified by the cooling water contained in the lower portion of the tub, as the discharge amount of the cooling water is increased, the drying time is relatively shortened and the power consumption rate and the water consumption rate are increased.

That is, in the rapid drying mode, the cooling water discharge cycle is shortened to shorten the drying time so that the power consumption rate and the water consumption rate are increased. In the power saving mode and the water saving mode, the cooling water discharge cycle is relatively lengthened so that the power consumption rate and the water consumption rate are decreased.

The cold air drying time is a time when the heater is switched off, the cooling water is not supplied to the washing and drying machine, and only the circulation fan is rotated, so that laundry in the drum is dried using heat remaining in the laundry. As the cold air drying time is increased, the power consumption rate is minimized and the drying time is increased.

That is, in the power saving mode, the cold air drying time is increased and the hot air drying time is relatively decreased so that the power consumption rate is minimized, in the rapid drying mode, the cold air drying time is decreased to shorten the drying time, and in the water saving mode, the cold air drying time is decreased to ensure a sufficient hot air drying time.

The supply and discharge of the cooling water and the cold air drying operation may be performed respectively in the rapid drying mode, the power saving mode, and the water saving mode according to a predetermined designated program or according to the temperature of the cooling water or the circulated air. Hereinafter, the supply and discharge of the cooling water and the cold air drying operation, which are performed according to the temperature of the cooling water or the circulated air, will be described.

The controller 140 controls the motor 90, the drainage pump 74, the heater 103, the fan motor 108, and the cooling water valve 132 according to the temperature (T) sensed by at least one of the tub temperature sensor 78, the heater air temperature sensor 112, and the condensed air temperature sensor 122.

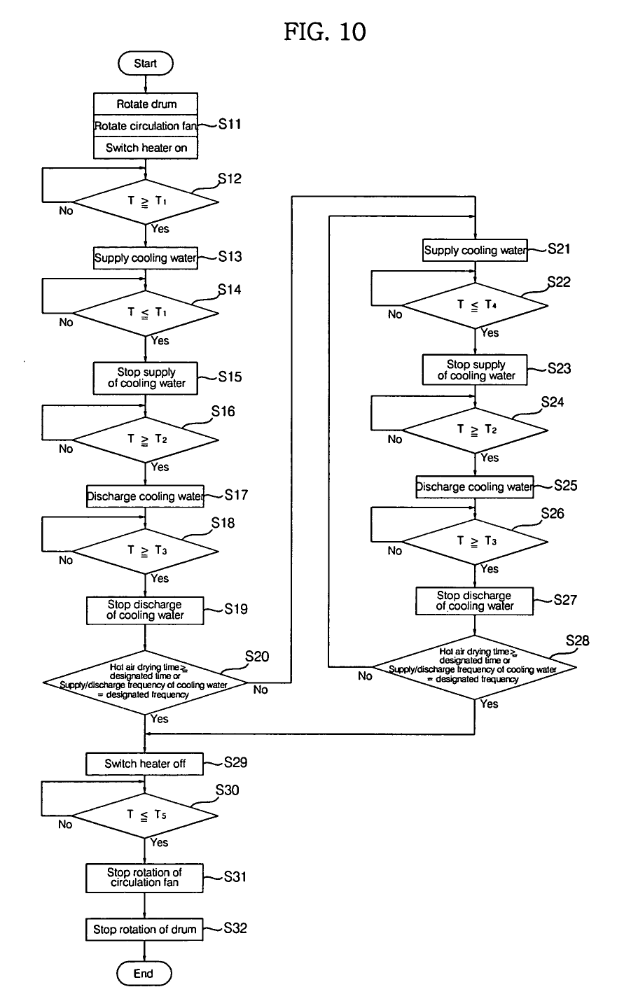

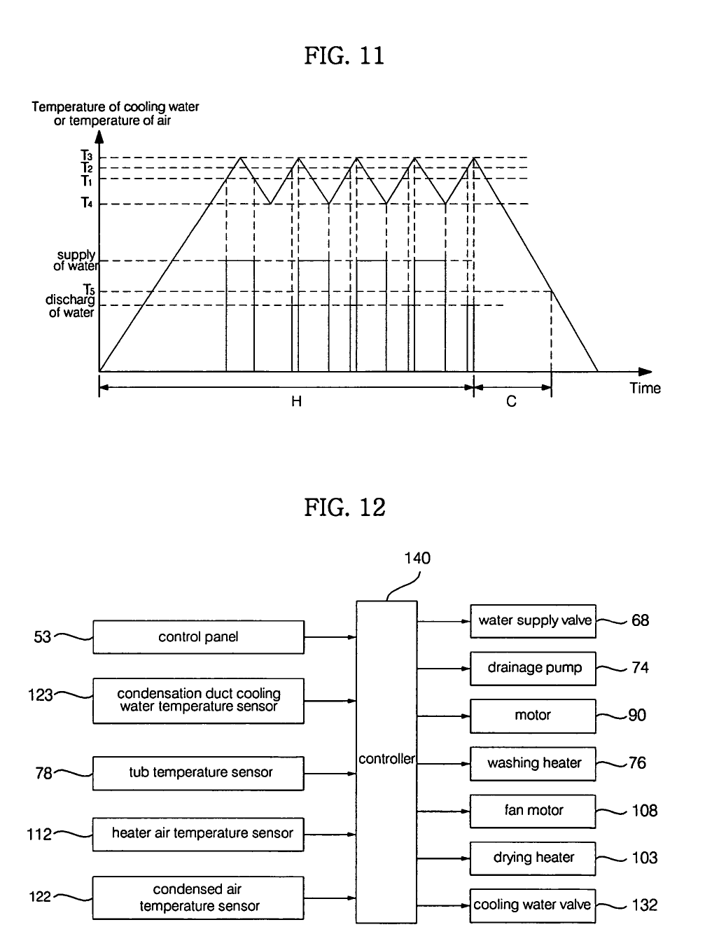

FIG. 10 is a flow chart illustrating the drying control method of the washing and drying machine in accordance with the second embodiment of the present invention, in one mode out of the rapid drying mode, the power saving mode, and the water saving mode. FIG. 11 is a graph illustrating cooling water supply/discharge times and temperature of the cooling water or circulated air according to the cold air drying operation when the drying control method in accordance with the second embodiment of the present invention is in one mode out of the rapid drying mode, the power saving mode, and the water saving mode.

The controller 140, as shown in FIG. 10, first performs the hot air drying operation (H), and then performs the cold air drying operation (C) after the hot air drying operation (H). When the cold air drying operation (C) is completed, the overall drying operation is terminated.

In the hot air drying operation (H), the controller 140, as shown in FIGS. 2 and 3, drives the motor 90 so that the drum 80 is rotated to agitated the laundry (m) in the drum 80, drives the fan motor 108 so that the circulation fan 107 is rotated to cause the air in the drum 80 to sequentially pass through the through holes 82 of the drum 80, the tub 60, the condensation duct 120, and the heater duct 110 and to be circulated into the drum 80, and switches the heater 103 on so that the air passing through the heater duct 110 is heated by the heater 103 (S11).

As shown in FIGS. 2 and 3, the air in the drum 80 moves towards the through holes 82 by the rotation of the circulation fan 107, contacts the laundry (m) agitated in the drum 80, and robs the laundry (m) of moisture, thereby being converted into a low-temperature and high-humidity state. After the air passes through the through holes 82, the air moves to a space between the outer cylindrical surface of the drum 80 and the inner cylindrical surface of the tub 60, and then passes through the condensation duct 120.

The air in the low-temperature and high-humidity state, having passed through the condensation duct 120, is introduced into the heater duct 110, and is heated by the heater 103, thereby being converted into hot air.

The hot air passes through the gasket 64, and is circulated into the drum 80 to rob the laundry (m) of moisture. By repeating the above circulation and heating, the temperature of the air is gradually increased, as shown in FIG. 11.

The controller 140 compares the temperature of the circulated air to the first designated temperature (T₁) in order to determine whether or not the cooling water is initially supplied to the washing and drying machine during the drying operation (S12).

Here, the controller 140 compares the temperature (T, hereinafter, referred to as the temperature of the circulated air), which is measured by one of the heater air temperature sensor 112 of the heater duct 110 and the condensed air temperature sensor 122 of the condensation duct 120, to the first designated temperature (T₁).

When the temperature (T) of the circulated air is more than the first designated temperature (T₁), the controller 140 switches the cooling water valve 132 on.

When the cooling water valve 132 is switched on, the air in the low-temperature and high-humidity state, which moves into the condensation duct 120, is robbed of heat by the cooling water supplied to the condensation duct 120, and is converted into a low-humidity state. Further, the temperature of the air is gradually decreased, as shown in FIG. 11.

The controller 140 re-compares the temperature (T) of the circulated air to the first designated temperature (T₁) in order to determine whether or not the supply of the cooling water is stopped during the drying operation (S14).

When the measured temperature (T) of the circulated air is less than the first designated temperature (T₁), the controller 140 switches the cooling water valve 132 off so that the supply of the cooling water is stopped (S15).

The cooling water supplied to the condensation duct 120, as shown in FIG. 3, drops towards the lower portion of the inside of the tub 60, and, even when the supply of the cooling water is stopped, the circulated air is continuously robbed of heat by the cooling water collected in the lower portion of the inside of the tub 60 so that moisture contained in the circulated air is condensed into water, and continuously dries the laundry.

As the drying operation is progresses, the temperature of the circulated air gradually decreases. When the temperature of the circulated air is the same as that of the cooling water due to the decrease in heat absorption rate/moisture condensation rate of the cooling water, as shown in FIG. 11, the temperatures of the cooling water and the circulated air are increased again.

The controller 140 compares the temperature (T) of the cooling water to the second designated temperature (T₂, here, T₂>T₁) in order to determine whether or not the cooling water is discharged to the outside of the washing and drying machine (S16).

That is, the controller 140 compares the temperature measured by the tub temperature sensor 78 (T, hereinafter, referred to as the temperature of the cooling water) to the second designated temperature (T₂).

When the temperature (T) of the cooling water is more than the second designated temperature (T₂), the controller 140 switches the drainage pump 74 on (S17).

When the drainage pump 74 is switched on, the cooling water collected in the lower portion of the inside of the tub 60 is discharged to the outside of the washing and drying machine through the drainage bellows 73, the drainage pump 74, and the drainage hose 75, and the temperature of the circulated air is continuously increased, as shown in FIG. 11.

The controller 140 compares the temperature of the circulated air to the third designated temperature (T₃, here, T₃>T₂) in order to determine whether or not the discharge of the cooling water is stopped (S18).

That is, the controller 140 compares the temperature (T, hereinafter, referred to as the temperature of the circulated air) measured by one of the heater air temperature sensor 112 of the heater duct 110 and the condensed air temperature sensor 122 of the condensation duct 120 to the third designated temperature (T₃).

When the temperature (T) of the circulated air is more than the third designated temperature (T₃), the controller 140 switches the drainage pump 74 off so that the discharge of the cooling water is stopped (S19).

When it is determined that the time taken to perform the above hot air drying operation (H) does not elapse a designated hot air drying time (for example, 80 minutes) or the supply/discharge frequency of the cooling water does not reach a designated frequency (for example, three times), the controller 140 switches the cooling water valve 132 on again for supplying cooling water, thereby re-supplying the cooling water to the washing and drying machine (S20 and S21).

When the cooling water is re-supplied to the washing and drying machine, the circulated air passed through the condensation duct 120 is robbed of heat by the re-supplied cooling water, and the temperature of the circulated air is decreased again, as shown in FIG. 11.

Thereafter, the controller 140 compares the temperature (T) of the circulated air to a fourth designated temperature (T₄, here, T₄<T₁) in order to determine whether or not the re-supply of the cooling water is stopped during the drying operation (S22).

When the measured temperature (T) of the circulated air is less than the fourth designated temperature (T₄), the controller 140 switches the cooling water valve 132 off so that the re-supply of the cooling water is stopped (S23).

The cooling water re-supplied to the condensation duct 120, as shown in FIG. 3, drops towards the lower portion of the inside of the tub 60, and, even when the supply of the cooling water is stopped, the circulated air is continuously robbed of heat by the cooling water collected in the lower portion of the inside of the tub 60 so that moisture contained in the circulated air is continuously condensed into water, and continuously dries the laundry.

When the temperature of the circulated air is the same as that of the cooling water, as shown in FIG. 11, the temperatures of the cooling water and the circulated air are increased again.

Thereafter, the controller 140 compares the temperature (T) of the cooling water to the second designated temperature (T₂) in order to determine whether or not the cooling water is re-discharged to the outside of the washing and drying machine, and, when the temperature (T) of the cooling water is more than the second designated temperature (T₂), switches the drainage pump 74 on so that the cooling water is re-discharged to the outside of the washing and drying machine (S24 and S25).

Then, the controller 140 compares the temperature of the circulated air to the third designated temperature (T₃) in order to determine whether or not the discharge of the cooling water is stopped, and, when the temperature (T) of the circulated air is more than the third designated temperature (T₃), switches the drainage pump 74 off so that the re-discharge of the cooling water is stopped (S26 and S27).

When it is determined that the time taken to perform the above hot air drying operation (H) does not elapse the designated hot air drying time (for example, 80 minutes) or the supply/discharge frequency of the cooling water does not reach the designated frequency (for example, three times), the controller 140 repeats the re-supply and re-discharge of the cooling water (S28, S21, S22, S23, S24, S25, S26, and S27).

On the other hand, when it is determined that the time taken to perform the above hot air drying operation (H) elapses the designated hot air drying time or the supply/discharge frequency of the cooling water reaches the designated frequency, the controller 140 switches the heater 103 off so that the cold air drying operation (C) is performed (S29).

In the cold air drying operation (C), the controller 140 switches the heater 103 off, and continuously rotates only the circulation fan 107. Then, the air in the drum 80 is circulated into the tub 60, the condensation duct 120, the heater duct 110, and the drum 80, thereby gradually decreasing the temperature of the laundry (m).

The controller 140 compares the temperature (T) measured by the heater air temperature sensor 112 to a fifth designated temperature (T₅, here, T₅<T₁) in order to adjust the cold air drying time according to the load, and, when the temperature (T) is less than the fifth designated temperature (T₅), switches the fan motor 108 off so that the circulation of the air in the drum 80 is terminated (S30 and S31).

Here, the fifth designated temperature (T₅) is a temperature for determining whether or not the cold air drying operation is terminated.

That is, when the load is small, it takes a short time to reach the fifth designated temperature (T₅), and when the load is large, it takes a long time to reach the designated fifth temperature (T₅). Accordingly, whether or not the cold air drying operation is terminated is determined by the fifth designated temperature (T₅). Thereby, when the load is small, the drying time is shortened, and when the load is large, the drying efficiency is increased and burns of consumers are prevented.

Thereafter, the controller 140 switches the motor 90 off, thereby terminating the overall drying operation including the cold air drying operation (S32).

In the rapid drying mode, the power saving mode, and the water saving mode, the controller 140 sets the first to fifth designated temperatures to different values, thereby maximally shortening the overall drying time, minimizing the power consumption rate, or minimizing the water consumption rate.

For example, in the rapid drying mode, the controller 140 sets the first designated temperature to a relatively low value so that the initial supply of the cooling water is performed early, sets the second and third designated temperatures to relatively low values so that the cooling water discharge cycle is shortened, sets the fourth designated temperature to a relatively high value so that the cooling water supply cycle is shortened, and sets the fifth designated temperature to a relatively high value so that the cold air drying time is shortened. The drying operation in the rapid drying mode is rapidly completed compared to the drying operation in the power saving mode or the water saving mode.

In the drying control method of the washing and drying machine in accordance with the second embodiment of the present invention, when one of the rapid drying mode, the power saving mode, and the water saving mode is inputted to the washing and drying machine, the drying operation is performed according to the inputted mode. Accordingly, the drying control method in accordance with the second embodiment can rapidly perform the drying operation, minimize the power consumption rate, or minimize the cooling water consumption rate according to user's desire, thereby increasing convenience in using the washing and drying machine.

Since the drying operation is performed according to drying conditions, which are set by the input of the kind, the drying time, and the dryness of laundry to be dried, and are differently modified by the inputted mode, the drying control method in accordance with the second embodiment performs the drying operation only when a user selects one of the rapid drying mode, the power saving mode, and the water saving mode.

Since the initial cooling water supply time, the cooling water supply cycle, the cooling water discharge cycle and the cold air drying time are set to different values in the rapid drying mode, the power saving mode, and the water saving mode, the drying control method in accordance with the second embodiment simply and rapidly adjusts the overall drying time, the power consumption rate, and the cooling water consumption rate.

Since the initial cooling water supply time, the cooling water supply cycle, the cooling water discharge cycle and the cold air drying time are changed using the temperature measured by at least one of the temperature sensor of the heater duct, the temperature sensor of the condensation duct, and the temperature sensor of the tub, the drying control method in accordance with the second embodiment spontaneously and rapidly copes with variation in load.

FIG. 12 is a control block diagram of a washing and drying machine, to which a drying control method in accordance with a third embodiment of the present invention is applied.

The washing and drying machine comprises the controller 140 serving as control means for receiving sensed results from the air temperature sensors 112 and 122 and the cooling water temperature sensors 78 and 123 and controlling the drying of laundry, such as the drying termination time of the laundry, according to the sensed results in the drying operation. The controller 140 may control the overall operation of the washing and drying machine. Preferably, the controller 140 is connected to the control panel 53 so as to input or output control signals to or from various control components, such as the water supply valve 68, the drainage pump 74, the motors 90 and 108, the heaters 76 and 103, the sensors 78, 112, 122 and 123, and the valve 132.

Hereinafter, the drying control method using the above washing and drying machine of the present invention will be described.

When the drying operation of the washing and drying machine is started, the motor 90 is operated by the control signal of the controller 140 so that the drum 80 is slowly rotated, the circulation fan 106 and the drying heater 103 in the heater duct 110 are operated, and the cooling water valve 132 is opened so that the cooling water is supplied to the inside of the condensation duct 120.

Accordingly, the air in the drum 80 passes through the condensation duct 120 and the heater duct 110 by the rotation of the circulation fan 106, and is circulated again to the inside of the drum 80. The air, which contacts the laundry in the drum 80, robs the laundry of heat and moisture, thereby being converted into a low-temperature and high-humidity state. Then, the air in the low-temperature and high-humidity state is introduced into the condensation duct 120, and when the air passes through the condensation duct 120, the moisture contained in the air is condensed by the cooling water so that the air is dehumidified. The air, having passed through the condensation duct 120, passes through the heater duct 110, and is heated by the heater 103, thereby being converted into hot air. The hot air is circulated again into the drum 80. The laundry in the washing and drying machine is dried by repeating the above process.

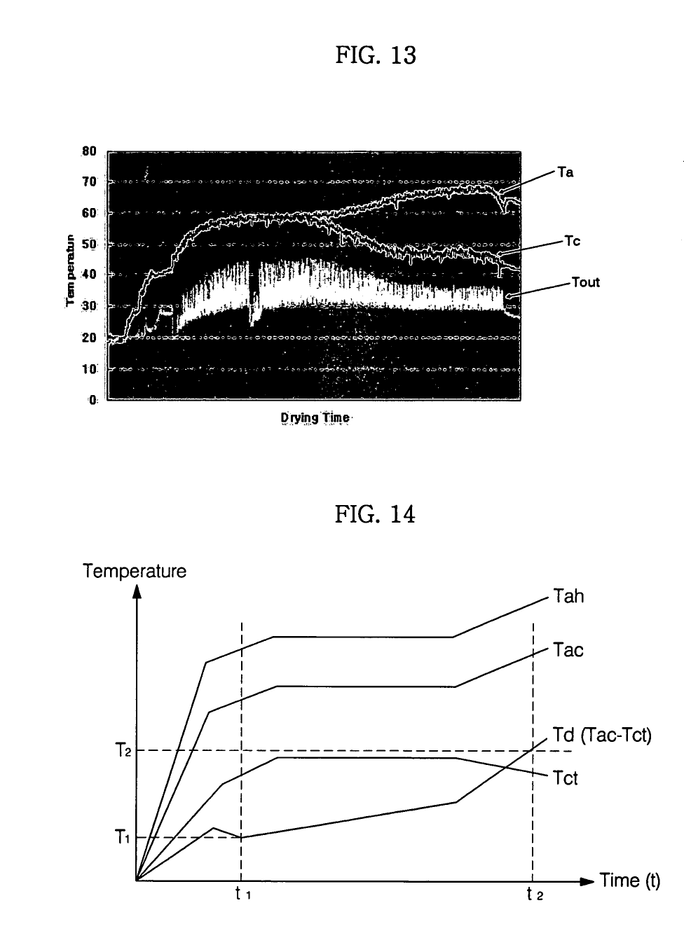

FIG. 13 is a graph illustrating temperature characteristics of air and cooling water in the drying control method in accordance with the third embodiment of the present invention, and FIG. 14 is a graph illustrating temperatures sensed by sensors and differences among the sensed temperatures in an initial stage of a drying operation in the drying control method in accordance with the third embodiment of the present invention.

In FIG. 13, Ta represents temperature characteristics of dehumidified air by passing through the condensation duct 120, and Tc represents temperature characteristics of cooling water dropped from the condensation duct 120 for dehumidifying circulation air. When the drying operation has progressed to some extent, a difference between the temperature (Ta) of the air and the temperature (Tc) of the cooling water is gradually increased.

Accordingly, the dryness is determined using the temperature characteristics of the air and the cooling water, and the drying operation is controlled by sensing the difference between the temperatures of the air and the cooling water.

For reference, Tout in FIG. 13 represents the temperature of the cooling water discharged to the outside of the tub 60.

First, after the drying operation for drying laundry is started, the temperature (Tc) of the cooling water, which condenses moisture contained in the air when the air drying the laundry passes through the condensation duct 120, and the temperature (Ta) of the air dehumidified by passing through the condensation duct 120 are measured.

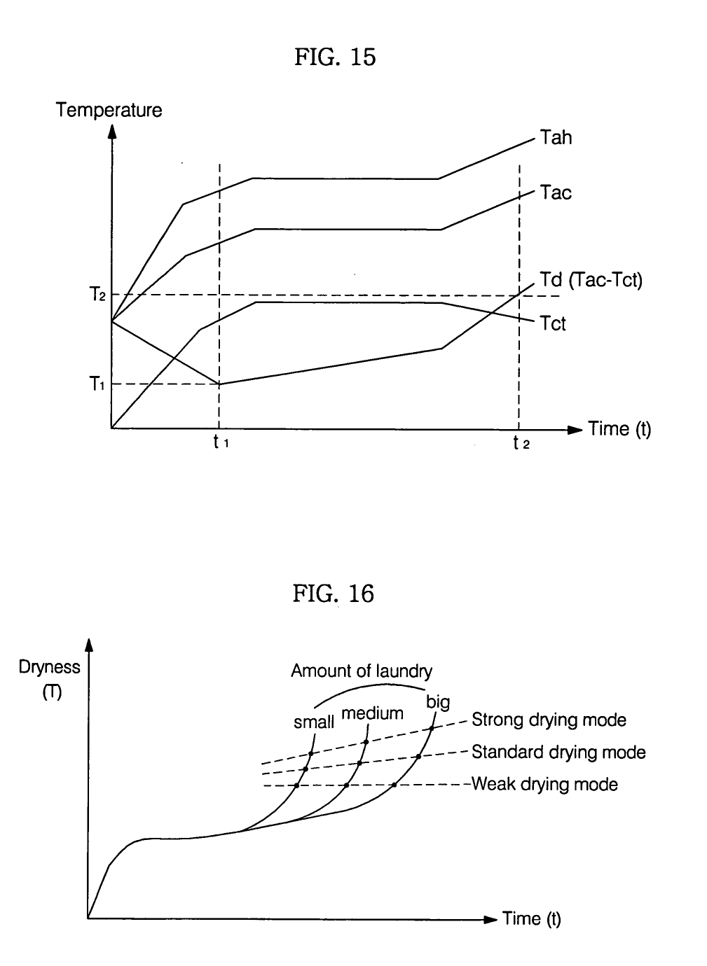

Here, as the temperature (Tc) of the cooling water, a temperature (Tcc) of the cooling water, which passes through the lower end of the condensation duct 120 and is sensed by the condensation duct cooling water temperature sensor 123 or 123′, or a temperature (Tct) of the cooling water, which is collected in the tub 60 and is sensed by the tub temperature sensor 78, may be measured. Alternately, both the temperature (Tcc) and the temperature (Tct) may be measured. Since the variation in the temperature (Tcc) of the cooling water sensed by the condensation duct cooling water temperature sensor 123 or 123′ and the variation in the temperature (Tct) of the cooling water sensed by the tub temperature sensor 78 are similar to each other, FIGS. 14 and 15 illustrates only the variation in the temperature (Tct) of the cooling water sensed by the tub temperature sensor 78.

As the temperature (Ta) of the air, a temperature (Tac) of the air, which is dehumidified by passing through the condensation duct 120 and is sensed by the condensed air temperature sensor 122, or a temperature (Tah) of the air, which passes through the heater 103 and is sensed by the heater air temperature sensor 112 may be measured. Since the variation in the temperature (Tac) of the air sensed by the condensed air temperature sensor 122 and the variation in the temperature (Tah) of the air sensed by the heater air temperature sensor 112 are similar to each other, one or all of the measured values of the two temperatures (Tac and Tah) of the air may be used. In this embodiment, the temperature (Tac) of the air sensed by the condensed air temperature sensor 122 is used.

When the drying operation for drying the laundry is continuously performed, the temperature (Tc) of the cooling water and the temperature (Ta) of the air are measured as described above. At this time, the dried state of the laundry is determined by a temperature difference (Td) between a signal representing the temperature (Tc) of the cooling water and a signal representing the temperature (Ta) of the air, thereby controlling the drying operation.

That is, for example, a time when the temperature difference (Td) is more than a designated value and reaches a predetermined determination temperature (T2), which is maintained more than a designated time, is set to a dying completion time (t2).

However, since drying characteristics of the laundry are varied according to the initial temperature of the cooling water and the temperature of peripheral air, it is preferable that initial operating conditions are compensated for by the below various methods in order to more precisely control the drying operation.

In the first method, the difference temperature (Td), the increase rate of which is less than a designated value after the drying operation is started, is set to an initial temperature (T1). As shown in FIG. 14, the temperature (Ta) of the air and the temperature (Tc) of the cooling water are increased at constant rates, and, the temperature (Ta) of the air is decreased after the drying of the laundry is substantially carried out. At this time, the curve of the difference temperature (Td) has an inflection point less than a designated value. The difference temperature (Td) at the inflection point is set to the initial temperature (T1).

In the second method, since the temperature of the air in the washing and drying machine is high after a boiling washing or a steam washing operation using the washing heater 76 of the washing and drying machine is carried out, or when a drying operation is started again just after the drying operation is completed, the difference temperature (Td) at a point of time when the difference temperature (Td) is decreased from the start of the drying operation and is then increased, i.e., when the increase rate of the difference temperature (Td) is less than a designated value, is set to the initial temperature (T1), as shown in FIG. 15.

In the third method, regardless of the increase rate of the difference temperature (Td) after the start of the drying operation, the difference temperature (Td) after a designated time (t1) from the start of the drying operation elapses is set to the initial temperature (T1). Preferably, the initial temperature (T1) is determined by appropriately selecting a point of time when the drying of the laundry is substantially carried out, through experimentation, which is repeated several times.

When the drying of the laundry to some extent is completed after the drying operation is continuously performed as described above, as shown in FIGS. 14 and 15, the temperature (Ta) of the air is rapidly increased and the temperature (Tc) of the cooling water is decreased. At this time, the difference temperature (Td) is rapidly increased, and reaches the predetermined determination temperature (T2).

Accordingly, the drying control method of the present invention determines the drying state of the laundry, i.e., the dryness of the laundry, through the difference between the determination temperature (T2) and the initial temperature (T1). Particularly, the drying control method of the present invention determines that a time when the difference between the determination temperature (T2) and the initial temperature (T1) is more than a designated value is the drying completion time (t2), and thus completes the drying operation.

Preferably, the individual temperature characteristics are determined through temperatures of the air and the cooling water when a target difference temperature value is continued more than a designated time so as to filter the variation in temperatures due to disturbance.

FIG. 16 is a graph illustrating the functional relation among amount of laundry, dryness of the laundry according to drying intensity, and time in the washing and drying machine in accordance with the third embodiment of the present invention. Preferably, the determination temperature (T2) is determined by the function among the intensity of the drying mode, the inputted drying time, and the water content of laundry to be dried (the amount of water contained in the laundry to be dried).

The drying modes include a strong drying mode, standard drying mode, and a weak drying mode. The greater the intensity of the drying mode, the higher the determination temperature (T2) is set.

Further, as the water content of the laundry to be dried is increased or as time goes by, the determination temperature (T2) may be set to a higher value.

The cooling water supplied to the condensation duct 120 passes through the condensation duct 120, is collected in the tub 60, and is periodically pumped out by the drainage device 74, thus, being discharged to the outside. Thereby, the drying operation is performed.

Since the dryness of the laundry to be dried is determined using a difference between the temperature of the dehumidified air and the cooling water dehumidifying the air so that the drying operation is controlled, the drying control apparatus and method of the washing and drying machine in accordance with the third embodiment of the present invention more precisely compensate for the dryness of the laundry to be dried according to load conditions, such as the temperature of the peripheral air and the temperature of the cooling water, and more efficiently control the drying operation, thereby improving the drying capability.

As apparent from the above description, the present invention provides a drying control apparatus and method of a washing and drying machine which enable a drying operation to be appropriately performed according to operating conditions.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

For example, although the preferred embodiments of the present invention have described a washing and drying machine, the drying control apparatus and method of the present invention may be applied to a drying machine, which has only a drying function.

What is claimed is:

1. A drying control method of a washing and drying machine, in which air in a drum is circulated so that the air sequentially passes through a tub, a condensation duct, and a heater duct, and is introduced again to the inside of the drum, and dries laundry in the drum, comprising:
supplying cooling water for condensing moisture contained in the circulated air to the condensation duct;
measuring a temperature of the cooling water dropped from the condensation duct to the tub, or a temperature of the circulated air; and
discharging the cooling water from the tub to the outside of the tub, when the measured temperature is more than a designated temperature.

2. The drying control method as set forth in claim 1, wherein, in the supplying of the cooling water, the cooling water is supplied to a level lower than the bottom of the drum.

3. The drying control method as set forth in claim 1, wherein a heater of the heater duct is switched on.

4. The drying control method as set forth in claim 3, wherein:
the heater is switched off in the last stage of a drying operation to perform a cold air drying operation; and
the circulation of the air in the drum is terminated when the temperature of the circulated air reaches a designated cold air drying termination temperature.

5. The drying control method as set forth in claim 3, wherein:
the drum is rotated; and
the rotation of the drum is terminated when the temperature of the circulated air is less than a designated cold air drying termination temperature.

6. A drying control method of a washing and drying machine comprising:
drying laundry in a drum using hot air; and
drying the laundry in the drum using cold air after the drying of the laundry using the hot air,
wherein, in the drying of the laundry using the cold air, the circulation of air is terminated when a temperature of the circulation air is less than a designated cold air drying termination temperature.

7. The drying control method as set forth in claim 6, wherein, in the drying of the laundry using the hot air,
air in the drum is circulated so that the air sequentially passes through a tub, a condensation duct, and a heater duct, and is introduced again to the inside of the drum;
a heater of the heater duct is switched on; and
cooling water for condensing moisture contained in the air is supplied to the condensation duct.

8. The drying control method as set forth in claim 6, wherein, in the drying of the laundry using the cold air,
air in the drum is circulated so that the air sequentially passes through a tub, a condensation duct, and a heater duct, and is introduced again to the inside of the drum;
a heater of the heater duct is switched off; and
cooling water for condensing moisture contained in the air is not supplied to the condensation duct.

9. The drying control method as set forth in claim 6, wherein:
the drum is rotated; and
the rotation of the drum is terminated when the temperature of the circulated air is less than a designated cold air drying termination temperature.

10. A drying control method of a washing and drying machine, which controls a circulation fan for causing air in a drum to sequentially pass through a tub, a condensation duct, and a heater duct and to be introduced again into the drum, a cooling water valve for supplying cooling water to the condensation duct so that moisture contained in the air passing through the condensation duct is condensed by the cooling water, and a heater for heating the air passing through the heater duct, wherein:
ON/OFF conditions of the circulation fan, the cooling water valve, and the heater are determined according to the input of kind, drying time, and dryness of laundry to be dried, and
when one mode out of a rapid drying mode, a power saving mode, and a water saving mode is inputted to the washing and drying machine, the ON/OFF condition of at least one of the circulation fan, the cooling water valve, and the heater is changed according to the inputted mode.

11. The drying control method as set forth in claim 10, wherein at least one of an initial cooling water supply time, a cooling water supply cycle, a cooling water discharge cycle, and a cold air drying time is changed in the rapid drying mode, the power saving mode, and the water saving mode.

12. The drying control method as set forth in claim 11, wherein the initial cooling water supply time in the rapid drying mode and the power saving mode is earlier than that in the water saving mode.

13. The drying control method as set forth in claim 11, wherein the cooling water supply cycle in the rapid drying mode is the shortest, and the cooling water supply cycle in the water saving mode is the longest.

14. The drying control method as set forth in claim 11, wherein the cooling water discharge cycle in the rapid drying mode is shorter than that in the power saving mode and the water saving mode.

15. The drying control method as set forth in claim 11, wherein the cold air drying time in the rapid drying mode and the water saving mode is shorter than that in the power saving mode.

16. The drying control method as set forth in claim 11, wherein at least one of the initial cooling water supply time, the cooling water supply cycle, the cooling water discharge cycle, and the cold air drying time is changed using a temperature measured by at least one of a temperature sensor of the heater duct, a temperature sensor of the condensation duct, and a temperature sensor of the tub.

17. The drying control method as set forth in claim 16, wherein:
the initial supply of the cooling water is performed during when the temperature measured by one of the temperature sensor of the heater duct and the temperature sensor of the condensation duct is more than a first designated temperature;
the discharge of the cooling water is performed when the temperature measured by the temperature sensor of the tub is more than a second designated temperature;
the supply of the cooling water after the initial supply is performed when the temperature measured by one of the temperature sensor of the heater duct and the temperature sensor of the condensation duct is more than a third designated temperature, and is stopped when the temperature measured by one of the temperature sensor of the heater duct and the temperature sensor of the condensation duct is less than a fourth designated temperature; and
the cold air drying operation is completed when the temperature measured by one of the temperature sensor of the heater duct and the temperature sensor of the condensation duct is less than a fifth designated temperature.

18. The drying control method as set forth in claim 17, wherein the first, second, third, fourth, and fifth designated temperatures are set to different values according to the rapid drying mode, the power saving mode, and the water saving mode.

19. A drying control apparatus of a washing and drying machine comprising:
a tub having a drum installed therein for containing laundry so that the laundry is washed or dried;
a condensation duct connected to one side of the tub for eliminating moisture contained in air drying the laundry by condensation;
a heater duct connected to the upper end of the condensation duct and the other side of the tub for heating the air having passed through the condensation duct and circulating the air into the tub;
an air temperature sensor for measuring a temperature of the air dehumidified by passing through the condensation duct;
a cooling water temperature sensor installed at the lower end of the tub or at an inlet of the condensation duct for measuring the temperature of cooling water; and
control means for receiving results sensed by the air temperature sensor and the cooling water temperature sensor and controlling the drying of the laundry using a difference between the temperature of the air and the temperature of the cooling water.

20. The drying control apparatus as set forth in claim 19, wherein:
the condensation duct has a cooling water chamber with the lower portion in which the cooling water is collected; and
the cooling water temperature sensor is located in the cooling water chamber.

21. A drying control apparatus of a washing and drying machine, in which:
when air drying laundry passes through a condensation duct after a drying operation for drying the laundry is started, a temperature of cooling water condensing moisture contained the air and a temperature of the air dehumidified by passing through the condensation duct are measured, and
when the drying operation is continued, the dried state of the laundry is determined by a difference temperature between a signal representing characteristics of the temperature of the cooling water and a signal representing characteristics of the temperature of the air, so as to control the drying operation.

22. The drying control apparatus as set forth in claim 21, wherein the temperature of the cooling water is measured by sensing at least one of a temperature of the cooling water, which passes through the lower end of the condensation duct, or a temperature of the cooling water, which is collected in a tub to which the condensation duct is connected.

23. The drying control apparatus as set forth in claim 21, wherein the temperature of the air is measured by sensing at least one of a temperature of the air, which is dehumidified by passing through the condensation duct, or a temperature of the air, which passes through a heater heating the air having passed through the condensation duct.

24. The drying control apparatus as set forth in claim 21, wherein a time when the difference temperature reaches a predetermined determination temperature is considered as a drying completion time.

25. The drying control apparatus as set forth in claim 21, wherein:
a temperature when the increase rate of the difference temperature after the drying operation is started is less than a designated value is referred to as an initial temperature; and
when, after the drying operation is continued, the difference temperature is continuously maintained more than the predetermined determination temperature for a designated time, a time when a difference between the initial temperature and the determination temperature is more than a designated value is considered as a drying completion time.

26. The drying control method as set forth in claim 21, wherein a time when a difference between an initial temperature and a predetermined determination temperature, when the increase rate of the difference temperature after the drying operation is started is less than a designated value, is more than a designated value is considered as a drying completion time.

27. The drying control method as set forth in claim 21, wherein a time when a difference temperature between an initial temperature and a predetermined determination temperature, after a designated time from the starting of the drying operation elapses, is more than a designated value is considered as a drying completion time.
